(19) United States
(12) Patent Application Publication
(10) Pub. No.: US 2023/0049162 A1
(43) Pub. Date: Feb. 16, 2023

(54) DEACTIVATION SOLUTION USEABLE FOR MICROORGANISM DETECTION

(71) Applicant: 3M INNOVATIVE PROPERTIES COMPANY, St. Paul, MN (US)

(72) Inventor: Sailaja Chandrapati, Woodbury, MN (US)

(21) Appl. No.: 17/790,527

(22) PCT Filed: Jan. 4, 2021
(86) PCT No.: PCT/IB2021/050019

§ 371 Date: Jul. 1, 2022

Related U.S. Application Data
(60) Provisional application No. 62/958,793, filed on Jan. 9, 2020.

(51) Int. Cl. C07D 307/62; C12N 1/38; C12N 1/20

(52) U.S. Cl. CPC C07D 307/62; C12N 1/38; C12N 1/205; C12N 2500/38; C12N 2500/35

(57) ABSTRACT

Composition useful for, among other things, culturing and enumerating microorganisms, and associated methods.

### BACKGROUND

US20130177938 discloses a neutralization broth for microorganism recovery. The broth does not include an ascorbic acid compound.

### DETAILED DESCRIPTION

Throughout this disclosure, singular forms such as “a,” “an,” and “the” are often used for convenience; however, the singular forms are meant to include the plural unless the singular alone is explicitly specified or is clearly indicated by the context. When the singular alone is called for, the term “one and only one” is typically used.

Some terms in this disclosure are defined below. Other terms will be familiar to the person of skill in the art, and should be afforded the meaning that a person of ordinary skill in the art would have ascribed to them.

Terms indicating a high frequency, such as (but not limited to) “common,” “typical,” and “usual,” as well as “commonly,” “typically,” and “usually” are used herein to refer to features that are often employed in the context of this disclosure and, unless specifically used with reference to the prior art, are not intended to mean that the features are present in the prior art, much less that those features are common, usual, or typical in the prior art.

The use of “deactivate” or its conjugates with regard to antimicrobial compounds means to reduce or eliminate the interference of antimicrobial compounds with culturing or detection of microorganisms.

“M” is used as an abbreviation for “molarity,” a unit of concentration. It may be preceded by standard prefixes indicating orders of magnitude, most typically “m” as in “mM,” for 10⁻³ molar.

Food producers, restaurants, pharmaceutical producers, and other industries require sampling of surfaces to determine the presence or absence, and in some cases quantification, of certain microorganisms. Generally, these industries will clean, sanitize, or clean and sanitize surfaces with antimicrobial agents, such as bleach, iodine, quaternary ammonium compounds, and the like. Testing the surfaces for the presence or absence, and in some cases quantification, or certain microorganisms can show whether cleaning or sanitation procedures are sufficient.

This disclosure recognizes that residual antimicrobial agents can interfere with the culturing or detection of microorganisms even when the microorganisms are not killed by the antimicrobial agents. The result is that false negatives may be observed wherein live microorganism of interest is present on the surface, but it is not detected in a culture because of the antimicrobial agents interfere with the culturing or detection. Such false negative can result in the mistaken belief that surfaces are sufficiently free of microorganisms where in fact the surfaces have microorganisms that can transfer to, for example, food products and cause sickness or death.

Thus, a problem to be solved is how to deactivate an antimicrobial agent in the presence of microorganisms so that one or more microorganisms not killed by the antimicrobial agent can be recovered and cultured. Another problem to be solved is how to culture an environmental sample of microorganisms when the sample includes antimicrobial agent. Yet another problem is how to reduce the instances of, or avoid, false negative results when culturing environmental samples.

An additional problem to be solved is to effectively deactivate any residual antimicrobial agents with components that do not adversely impact downstream detection assays. It is not necessarily sufficient that the microorganisms can be recovered and cultured. It may also be necessary to detect, and in some cases enumerate, any microbes that are collected from a surface. Thus, the deactivation of antimicrobials is, in optimal cases, done in such a way that the antimicrobials do not interfere with assays that may be used to detect or enumerate microorganisms after culturing. Also, in optimal cases, the substances used for deactivation will not themselves interfere with assays that may be used to detect or enumerate microorganisms after culturing.

Ascorbic acid compounds, particularly those selected from ascorbic acid, 2-phospho ascorbic acid, 6-O-alkyl ascorbic acid, 2,6-di-O-alkenyl ascorbic acid, 3-0 alkyl ascorbic acid, 2-O-α-glycopyranosyl ascorbic acid, glyceryl ascorbate, 5,5-di-O-alkyl ascorbic acid, at least one salt form of any of the foregoing, and at least one hydrate of any of the foregoing, have some uses in food science, but are known to degrade rapidly and are not known to be useful for deactivating residual antimicrobials or in reducing instances of false negative results when culturing environmental samples Thus, more specific versions of the problem to be solved is how to use ascorbic acid compounds, particularly those mentioned immediately above, to deactivate residual antimicrobials, reduce instances of false negative results when culturing environmental samples, or both. A variation of this problem is how to slow the degradation of ascorbic acid compounds, such as those mentioned above and particularly ascorbic acid, such that the ascorbic acid can be effective after storage.

The inventors have recognized that polysorbate may cause difficulties in controlling the pH of compositions for deactivating antimicrobials even when a buffer is used. Thus a related problem, which is solved by some but not necessarily all of the embodiments of the present disclosure, is how to deactivate residual antimicrobials, reduce instances of false negative results when culturing environmental samples, or both, without the use of polysorbates.

A related problem, which is solved by some but not necessarily all of the embodiments of the present disclosure, is how to use ascorbic acid compounds, particularly those mentioned above, to deactivate residual antimicrobials, reduce instances of false negative results when culturing environmental samples, or both, while avoiding the use of polysorbates.

A solution lies in the use of a composition as described herein, for example according to the methods described herein, to deactivate antimicrobial agents. Briefly, the composition comprises an ascorbic acid compound, at least one compound capable of deactivating an antimicrobial agent, a pyruvate, one or more buffers, and sufficient water to form a solution of all of the components of the composition. Particular examples of the composition do not contain polysorbate, because polysorbate can make it difficult to control the pH of the composition even when a buffer is used. Control of pH is important because in particular embodiments of the solution, the composition is in the form of a solution. In any of the above-mentioned embodiments of the solution, the composition can have a pH of about 6-8.

In any embodiment of the solution, the ascorbic acid compound can be any compound having the structure of ascorbic acid as part of its chemical structure. Particularly, in any embodiment described herein, the ascorbic acid compound is selected from ascorbic acid, 2-phospho ascorbic acid, 6-O-alkyl ascorbic acid, 2,6-di-O-alkenyl ascorbic acid, 3-O alkyl ascorbic acid, 2-O-α-glycopyranosyl ascorbic acid, glyceryl ascorbate, 5,5-di-O-alkyl ascorbic acid, at least one salt form of any of the foregoing, and at least one hydrate of any of the foregoing. More particularly, ascorbic acid, 2-phospho ascorbic acid, at least one salt form of any of the foregoing, or at least one hydrate of any of the foregoing are used in any embodiment. Most particularly 2-phospho ascorbic acid, at least one salt form thereof, or at least one hydrate of any of the foregoing, can be used in any embodiment.

In any of the aforementioned embodiments, the ascorbic acid compound can be used in any suitable amount. Suitable amounts include those that are sufficient to enable the at least one compound capable of deactivating an antimicrobial agent to deactivate an antimicrobial agent in an environment of interest. Thus, the amount may vary somewhat depending on the desired application. Particularly, the amount of ascorbic acid compound based on the weight of ascorbic acid and expressed in g/L can be, in particular examples, 0.001-20. More particularly, with regard to the minimum amount, the ascorbic acid compound, in g/L and based on the weight of ascorbic acid, in any embodiment can be 0.01 g/L or greater, 0.05 g/L or greater, 0.1 g/L or greater, 0.5 g/L or greater, 1 g/L or greater, 5 g/L or greater, 7.5 g/L or greater, 10 g/L or greater, or even 15 g/L or greater. More particularly, with regard to the maximum amount of ascorbic acid compound, the ascorbic acid compound, in g/L and based on the weight of ascorbic acid, in any embodiment can be 20 or less, 15 or less, 10 or less, or 5 or less. 0

The amount of ascorbic acid compound is expressed herein based on the weight of ascorbic acid. When an ascorbic acid compound other than ascorbic acid is used, the amount can be determined by comparing the molecular weight of the ascorbic acid compound that is employed to the molecular weight of ascorbic acid and making an appropriate calculation. The artisan will understand how to do this.

In any of the aforementioned embodiments, the at least one compound capable of deactivating an antimicrobial agent can include any compound suitable for this purpose. Importantly, in any embodiment, the at least one compound capable of deactivating an antimicrobial agent need not be capable of deactivating an antimicrobial agent in the absence of the other components of the composition described herein so long as it has this capability when used in the disclosed or claimed compositions. Particular examples of compounds that are capable of deactivating an antimicrobial agent include letheen broth, thioglycolates, such as sodium thioglycolate, one or more peptones, thiosulfates, such as sodium thiosulfate, and bisulfates, such as sodium bisulfates, protease, urea, phenol, calcium, collagen, haematin, melanin, polysaccharides, humic acids, and tannic acid. Some of the aforementioned compounds that are capable of deactivating an antimicrobial agent, specifically thiosulfates and the like, can interfere with some downstream detection assays. Thus, while such compounds may be used, in more particular aspects of any of the aforementioned embodiments, the at least one compound capable of deactivating an antimicrobial agent is selected from letheen broth, thioglycolates, such as sodium thioglycolate, one or more peptones, protease, urea, phenol, calcium, collagen, haematin, melanin, polysaccharides, humic acids, tannic acid and bisulfates, such as sodium bisulfate. In even more particular aspects of any of the aforementioned embodiments, the at least one compound capable of deactivating an antimicrobial agent is particularly selected from letheen broth, thioglycolates, such as sodium thioglycolate, one or more peptones, protease, urea, phenol, calcium, collagen, haematin, melanin, polysaccharides, humic acids, tannic acid and bisulfates, such as sodium bisulfate.

Most particularly, in any aforementioned embodiment, one or more peptones can be employed as the at least one compound capable of deactivating an antimicrobial agent. Any suitable peptone or combination of peptones can be used. Particularly, in any embodiment, the one or more peptones comprise one or more of soytone, tryptone, and porcine peptone, more particularly a combination of soytone, tryptone, and porcine peptone. Most particularly, the one or more peptones are a combination of soytone, tryptone, and porcine peptone.

In any embodiment in which they are employed, the amount of the at least one compound capable of deactivating an antimicrobial agent is an amount sufficient to deactivate the antimicrobial agent of interest in the environment of interest, and so it may depend somewhat on the intended use. In any of the aforementioned embodiments in which one or more peptones are employed as a compound capable of deactivating an antimicrobial agent, of the amount of the one or more peptones, in g/L, can be 0.001 or more, 0.005 or more, 0.01 or more, 0.05 or more, 0.1 or more, 0.25 or more, or 0.5 or more, 0.75 or more, 1 or more, 1.25 or more, 1.5 or more, 1.75 or more, 2 or more, 2.25 or more, 2.5 or more, 2.75 or more or even 3 or more. In any of the aforementioned embodiments in which one or more peptones are employed as a compound capable of deactivating an antimicrobial agent, of the amount of the one or more peptones, in g/L, can be 5 or less, 4.75 or less, 4.5 or less, 4.25 or less, 4 or less, 3.75 or less, 3.5 or less, 3.25 or less, 3 or less, 2.75 or less, 2.5 or less, 2.25 or less, 2 or less, 1.75 or less, 1.5 or less, 1.25 or less, 1 or less, 0.75 or less, 0.5 or less, 0.25 or less, 0.1 or less, or even 0.05 or less.

It should be noted that, in all embodiments, the compound capable of deactivating an antimicrobial agent is a component that is distinct from the other components of the composition. For example, even if pyruvate or ascorbic acid compounds may be capable of deactivating an antimicrobial agent in some circumstances, in this disclosure neither the pyruvate nor the ascorbic acid compound are considered to be the compound capable of deactivating an antimicrobial agent.

In any of the aforementioned embodiments, the pyruvate is most commonly in the form of sodium pyruvate, but as the composition is in the form of a solution other pyruvate salts can be used or can be present in the solution so long as the counter ion or counter ions of the pyruvate salt do not interfere with the desired properties of the composition. The potassium salt of pyruvate is one exemplary salt that may be used in any embodiment instead of or in combination with of sodium pyruvate.

The pyruvate is used in sufficient quantities to provide the desired deactivating effect. Particular amounts of pyruvate, in g/L based on the weight of sodium pyruvate, for any of the aforementioned embodiments are 1 or more, 2 or more, 3 or more, 4 or more, 5 or more, 6 or more, 7 or more, 8 or more, 9 or more, or even 10 or more. Particular amounts of pyruvate in any embodiment, in g/L, are 20 or less, 19 or less, 18 or less, 17 or less, 16 or less, or even 15 or less. More particularly, the amount of pyruvate can be about 1-20 g/L, optionally about 3-15 g/L, in each case based on the weight of sodium pyruvate.

Note that the all of amounts of pyruvate discussed here are based on the weight of sodium pyruvate. If a different pyruvate is used, the artisan can determine the correct amount by comparing the molecular weight of the pyruvate that is used with that of sodium pyruvate and making an appropriate calculation to account for the difference.

In any of the aforementioned embodiments, the one or more buffers can be any buffers so long the composition is at the requisite pH and resists changes in pH that preven successful culturing or detection of microorganisms. Particulalry, the concentration of the buffer, in any embodiment, can be 10 mM or greater, 15 mM or greater, 25 mM or greater, 30 mM or greater, 35 mM or greater, 40 mM or greater, 45 mM or greater, 50 mM or greater, 55 mM or greater, 60 mM or greater, 60 mM or greater, 65 mM or greater, 70 mM or greater, or even 75 mM or greater. Particularly, the concentration of the buffer, in any embodiment, can be 100 mM or less, 90 mM or less, 80 mM or less, 75 mM or less, 70 mM or less, 65 mM or less, 60 mM or less, 55 mM or less, 50 mM or less, 45 mM or less, 40 mM or less, 35 mM or less, or even 30 mM or less.

In any embodiment, buffers that can be used include buffers that are compatible with physiological systems, such as those that are compatible with culturing and detecting microorganisms. Particular examples include tris buffers, phosphate buffers, citrate buffers, acetate buffers, and the like. Particularly a phosphate buffer is employed. More particularly, the phosphate buffer is employed in addition to the one or more peptones.

In any of the previously mentioned embodiments, the composition may avoid the use of polysorbates, and particularly avoid the use of polysorbate surfactants, which are available under the trade designation TWEEN. Thus, in particular cases, the composition is free of polysorbate.

In any of the foregoing embodiments, the composition is in the form of a solution or dispersion and has sufficient water to dissolve or disperse all of the components. In any embodiment, the solution or dispersion particularly has a pH of about 6-8. The pH of a dispersion refers to the pH of the aqueous component of the dispersion. The pH can be provided by selecting the amounts and types of buffers that impart this pH and following the guidelines in this disclosure.

The composition, in any of the above embodiments, may also include one or more polyol surfactants. Particularly polyol surfactants that can be employed include glycerol, polyvinyl alcohol, and low molecular weight polymeric polyols, particularly polyether polyols, and the like. Most particularly glycerol is the most commonly employed polyol surfactant.

The composition may, in any of the foregoing embodiments, also comprise an emulsifier. Any emulsifier that does not interfere with the desired properties of the composition can in principle be employed. The emulsifier, if employed, is used in any embodiment an amount sufficient to create a dispersion of all of the components of the composition. A particular emulsifier, which may be employed in any embodiment, is lecithin, which is most commonly employed because it may also aid in deactivating one or more antimicrobial agents. In this description, however, lecithin is not considered to be an at least one compound capable of deactivating an antimicrobial agent.

In any of the foregoing embodiments that include an emulsifier, it can be used in any suitable amount to ensure that that the components of the composition are suitably dissolved or dispersed. Typical amounts of emulsifier, particularly lecithin, in g/L that can be employed in any embodiment are 1 or more, 2 or more, 3 or more, 4 or more, 5 or more, 6 or more, 7 or more, 8 or more, 9 or more, or even 10 or more. Typical amounts of emulsifier, particularly lecithin, in g/L that can be employed in any embodiment, are 20 or less, 19 or less, 18 or less, 17 or less, 16 or less, or even 15 or less. In particular cases about 1-20 g/L, optionally about 3-10 g/L, of emulsifier, most particularly lecithin, can be used.

The compositions as described herein surprisingly provide enhanced stability for the ascorbic acid compound. Ascorbic acid compounds are known in the art to degrade very rapidly, such as within a few hours, a few minutes, or even less, when they are dissolved or dispersed in water. The ascorbic acid compounds in the compositions described herein have significantly enhanced stability. Thus, any of the embodiments described herein may have the property that after storage at 2-8 degrees C. for 1 week under ambient atmospheric conditions, the amount of ascorbic acid compound in the composition decreases by no more than 60%, no more than 55%, no more than 50% no more than 45% no more than 40%, no more than 35%, no more than 30%, no more than 35%, no more than 30%, no more than 25%, or even no more than 20%, when compared to the amount of ascorbic acid compound present in the composition prior to storage at 2-8 degrees C. for 1 week.

It is notable that the combination of components in the compositions described herein have a synergistic effect in deactivating antimicrobial agents and allowing growth of microorganisms that would otherwise be inhibited by the presence of antimicrobial agents. For example, and as illustrated in the examples, absent the presence of the ascorbic acid compound the compositions may not be effective at deactivating some antimicrobial agents.

In use, a method of culturing one or more microorganisms can include sampling a surface to remove a sample of the one or more microorganisms from the surface. Surfaces can be sampled by a variety of techniques, for example, by using 3M™ DRY-SPONGES, available from 3M Company of St Paul, Minn., USA.

After sampling a surface, the sample can be contacted with a composition as described herein. The sample can be contacted with a growth media and at least some of the one or more microorganisms can be allowed to multiply within the growth media. The step of contacting the sample with a composition as described herein can take place before or after the sample is contacted with the growth medium. The amount of composition used is typically between 2 and 25 mL, particularly approximately 10 mL but can vary depending on the nature of the sample, the type of microorganism, and the nature and amount of antimicrobial agent that may be present on the surface.

The growth medium can be any growth medium. Thin film growth media, such as those available under the trade designation PETRIFILM® from 3M Company of St Paul, Minn., USA are preferred. However, the compositions and methods described herein are also compatible with agar-based media.

The microorganism that is cultured can be any microorganism. Most commonly, the microorganism is E. coli, Salmonella, typhimurium, Listeria monocytogenes, a Staphylococcus aureus microorganism, or a mixture thereof. A single type of microorganism can be cultured, for example by using a medium with selection agents that inhibit the growth of microorganisms other than the target microorganism, or more than one type of microorganism can be cultured.

### Examples

### Materials

TABLE 1

| Materials | Source |
|---|---|
| Polysorbate 80 | product #P1754; Sigma-Aldrich |
| (polyoxyethylene sorbitan | Corporation, St. Louis, MO |
| monooleate) | |
| Soy Lecithin (non-GMO, deoiled | Clarkson Soy Products, LLC, Cero |
| powder) | Gordo, IL |
| Sodium phosphate dibasic | VWR International, Radnor, PA |
| Potassium phosphate monobasic | product #PX1565; EMD Millipore, |
| | Burlington, MA |
| Sodium pyruvate | product #792500; Sigma-Aldrich |
| | Corporation |
| Porcine peptone | Alpha Biosciences, Baltimore, MD |
| Soytone (BACTO) | product #243620; Becton, Dickinson, |
| | Franklin Lakes, NJ |
| Tryptone | product #T7293; Sigma-Aldrich |
| | Corporation |
| Ascorbic acid | product #A92902; Sigma-Aldrich |
| | Corporation |
| 2-Phospho-L-ascorbic acid | product #49752; Sigma-Aldrich |
| trisodium salt | Corporation |
| Glycerol | product #G9012; Sigma-Aldrich |
| | Corporation |
| 2,6-dichlorophenolindophenol | product #103028, Sigma-Aldrich |
| sodium salt dihydrate (DCPIP) | Corporation |

Listeria monocytogenes ATCC 7644 (product #0398P) and Salmonella enterica subsp. enterica serovar Typhimurium ATCC 51812 (product #0180P) were obtained as KWIK-STIK kits from MICROBIOLOGICS Incorporated (St. Cloud, Minn.) and propagated per the manufacturer's instructions. Each KWIK-STIK kit was activated per the manufacturer's instructions. Each resulting saturated swab was streaked onto a separate, sterile tryptic soy agar plate [tryptic soy broth (TSB, product #211825) and agar (product #214010) were obtained from Becton, Dickinson] and incubated for 24-48 hours at 35° C. to obtain isolated colonies. Isolated colonies were transferred into individual sterile culture tubes containing 5 mL of TSB and incubated for 24 hours at 35° C. Escherichia coli (FSD723) was prepared from frozen stocks (−80° C.) by propagating in TSB for 24 hours at 35^{□}C. The individual TSB cultures were further diluted as needed in Butterfields buffer (product #BPPFV9BFD, 3M Company) to result in 100-500 colony forming units/milliliter (cfu/mL).

The Quat (quaternary ammonium) based antimicrobial agent solution was obtained as CHLOROX Broad Spectrum Quaternary Disinfectant Cleaner (the CHLOROX Company, Oakland, Calif.) and used as provided by the manufacturer (active ingredients reported by the manufacturer: 0.105% n-alkyl dimethyl benzyl ammonium chlorides and 0.105% n-alkyl dimethyl ethylbenzyl ammonium chlorides).

The hydrogen peroxide based antimicrobial agent solution was obtained as OXIVIR Five 16 concentrate (Diversey, Fort Mill, S.C.) and diluted 1:50 in sterile, distilled water prior to use.

Star San sanitizer, an acid based antimicrobial agent solution, was obtained from Five Star Chemicals & Supply (Commerce City, Colo.) and diluted 1:25 in sterile, distilled water prior to use. Before dilution the active ingredients in Star San sanitizer were reported to be 15% dodecylbenzene sulfonic acid and 50% phosphoric acid.

The bleach based antimicrobial agent solution (active ingredient 5% sodium hypochlorite) was obtained as product # LC246302 from LabChem (Zelienpole, Pa.) and diluted 1:50 in sterile, distilled water prior to use.

Rapicide Pa., a peracetic acid based antimicrobial agent solution (active ingredients as reported by the manufacturer: 22% hydrogen peroxide and 5% peracetic acid), was obtained from Medivators (Minneapolis, Minn.) and diluted 1:100 in sterile, distilled water prior to use.

### Examples 1-3 (EX 1-EX 3) and Comparative Examples A-F (CE A-CE F)

Compositions of the disclosure (Examples 1-3 and Comparative Examples A-F) were individually prepared by mixing in sterilizable glass containers the ingredients shown in Tables 2-4. The compositions were sterilized by autoclaving at 121° C. for 15 minutes and then stored in a refrigerator (2-8° C.) until used.

TABLE 2
Aqueous Compositions (Examples 1
& 2 and Comparative Example A)

| | Amounts of Ingredients | | | | | |
|---|---|---|---|---|---|---|
| | in the Compositions | | | | | |
| | | | | | Comparative | |
| | Example 1 | | Example 2 | | Example A | |
| Ingredients | (EX 1) | | (EX 2) | | (CE A) | |
| Soy Lecithin | 7 | g | 7 | g | 7 | g |
| Sodium phosphate dibasic | 12 | g | 12 | g | 12 | g |
| Potassium phosphate monobasic | 3 | g | 3 | g | 3 | g |
| Sodium pyruvate | 7.5 | g | 7.5 | g | 7.5 | g |
| Porcine peptone | 0.4 | g | 0.8 | g | 0.8 | g |
| Soytone | 0.4 | g | 0.8 | g | 0.8 | g |
| Tryptone | 0.4 | g | 0.8 | g | 0.8 | g |
| Ascorbic acid | 1 | g | 1 | g | 0 | g |
| Glycerol | 7.5 | mL | 7.5 | mL | 7.5 | mL |
| Distilled water | 992.5 | mL | 992.5 | mL | 992.5 | mL |

TABLE 3
Aqueous Composition (Example 3)

| | Amounts of Ingredients in the | |
|---|---|---|
| Ingredients | Composition of Example 3 (EX 3) | |
| Soy Lecithin | 7 | g |
| Sodium phosphate dibasic | 15 | g |
| Potassium phosphate monobasic | 3 | g |
| Sodium pyruvate | 7.5 | g |
| Porcine peptone | 0.8 | g |
| Soytone | 0.8 | g |
| Tryptone | 0.8 | g |
| 2-Phospho-L-ascorbic acid | 5 | g |
| trisodium salt | | |
| Glycerol | 7.5 | mL |
| Distilled water | 992.5 | mL |

TABLE 4
Aqueous Compositions (Comparative Examples B-F)

| | Amounts of Ingredients in the Compositions | | | | | | | | | |
|---|---|---|---|---|---|---|---|---|---|---|
| | Comparative | | Comparative | | Comparative | | Comparative | | Comparative | |
| | Example B | | Example C | | Example D | | Example E | | Example F | |
| Ingredients | (CE B) | | (CE C) | | (CE D) | | (CE E) | | (CE F) | |
| Polysorbate 80 (mL) | 5 | mL | 0 | mL | 0 | mL | 0 | mL | 0 | mL |
| Soy Lecithin | 7 | g | 7 | g | 0 | g | 0 | g | 0 | g |
| Sodium phosphate | 10 | g | 12 | g | 12 | g | 12 | g | 12 | g |
| dibasic | | | | | | | | | | |
| Potassium phosphate | 3 | g | 3 | g | 3 | g | 3 | g | 3 | g |
| monobasic | | | | | | | | | | |
| Sodium pyruvate | 0 | g | 10 | g | 0 | g | 10 | g | 0 | g |
| Porcine peptone | 0 | g | 0 | g | 0 | g | 0 | g | 0 | g |
| Soytone | 2.5 | g | 2.5 | g | 2.5 | g | 0 | g | 0 | g |
| Tryptone | 0 | g | 0 | g | 0 | g | 2.5 | g | 2.5 | g |
| Ascorbic acid | 1 | g | 0 | g | 1 | g | 0 | g | 1 | g |
| Glycerol | 5 | mL | 10 | mL | 10 | mL | 10 | mL | 10 | mL |
| Distilled water | 990 | mL | 990 | mL | 990 | mL | 990 | mL | 990 | mL |

### Example 4 (EX 4)

Culture tubes containing freshly prepared sterile, aqueous compositions of Example 1, Example 2, Example 3, and Comparative Examples A-F were prepared. Each tube contained 4.5 mL of a single aqueous composition. An aliquot (0.5 mL) of the Quat based antimicrobial solution (described above) was added by pipette to each tube and the tubes were shaken for 10-30 minutes to mix the contents. Corresponding control tubes were also prepared which contained 0.5 mL of added sterile water in place of the Quat based antimicrobial solution. Each tube was then inoculated with an aliquot of a single bacterial suspension selected from L. monocytogenes, S. typhimurium, or E. coli (each inoculum prepared as described above at a concentration of about 100-500 cfu/mL). The inoculated tubes were incubated at room temperature for one hour. Following the incubation period, two 1 mL samples was removed from each tube and each sample was individually plated onto a separate 3M PETRFILM Rapid Count Plate (3M Company, St. Paul, Minn.) according to the manufacturer's instructions. The plates were then incubated at 35° C. for 24 hours and the resulting colony forming units (cfu) per milliliter on each plate were enumerated by counting. The percent recovery of the bacteria was calculated by comparing the mean cfu/mL recovered (n=2) from tubes with the aqueous compositions of Example 1, Example 2, Example 3 and Comparative Examples A-F to the mean cfu recovered from the corresponding control tubes. The equation used to calculate percent bacteria recovery is provided in Equation 1. The results are presented in Tables 5-7.

$$\% Recovery = \frac{\begin{matrix}[ mean cfu count using \\ composition of the Example \times 100 ]\end{matrix}}{[ mean cfu count using control solution ]} \qquad Equation 1$$

### Example 5 (EX 5)

The same procedure as described in Example 4 was followed with the exception that the aliquot (0.5 mL) of Quat based antimicrobial solution was replaced with 0.5 mL of the diluted hydrogen peroxide based antimicrobial agent solution described above. The results are presented in Tables 5-7.

### Example 6 (EX 6)

The same procedure as described in Example 4 was followed with the exception that the aliquot (0.5 mL) of Quat based antimicrobial solution was replaced with 0.5 mL of the diluted acid based antimicrobial agent solution described above. The results are presented in Tables 5-7.

### Example 7 (EX 7)

The same procedure as described in Example 4 was followed with the exception that the aliquot (0.5 mL) of Quat based antimicrobial solution was replaced with 0.5 mL of the diluted bleach based antimicrobial agent solution described above. The results are presented in Tables 5-7.

### Example 8 (EX 8)

The same procedure as described in Example 4 was followed with the exception that the aliquot (0.5 mL) of Quat based antimicrobial solution was replaced with 0.5 mL of the diluted peracetic acid based antimicrobial agent solution described above. The results are presented in Tables 5-7.

TABLE 5

| | Percent Recovery of L. monocytogenes (equation 1) | | | | |
|---|---|---|---|---|---|
| | Type of Antimicrobial Solution Used | | | | |
| | | Hydrogen | | | |
| Aqueous | Quat | Peroxide | Acid | Bleach | Peracetic |
| Composition of | Based | Based | Based | Based | Acid Based |
| EX 1 | 70.8 | 74.5 | 54.7 | 78.9 | 87.0 |
| EX 2 | 72.7 | 55.7 | 35.2 | 65.3 | 79.5 |
| EX 3 | 104.4 | 102.2 | 81.1 | 86.1 | 112.8 |
| CE A | 48.3 | 27.3 | 0.0 | 0.0 | 7.9 |
| CE B | 6.3 | 0.0 | 47.8 | 71.4 | 0.0 |
| CE C | 19.8 | 0.0 | 59.0 | 3.0 | 0.8 |
| CE D | 23.1 | 0.5 | 41.8 | 68.4 | 0.5 |
| CE E | 58.1 | 5.0 | 63.3 | 4.2 | 32.5 |
| CE F | 31.4 | 11.3 | 44.0 | 76.0 | 7.3 |

TABLE 6

| | Percent Recovery of S. typhimurium (equation 1) | | | | |
|---|---|---|---|---|---|
| | Type of Antimicrobial Solution Used | | | | |
| | | Hydrogen | | | |
| Aqueous | Quat | Peroxide | Acid | Bleach | Peracetic |
| Composition of | Based | Based | Based | Based | Acid Based |
| EX 1 | 109.1 | 108.0 | 93.2 | 97.2 | 100.6 |
| EX 2 | 98.8 | 100.0 | 109.9 | 87.7 | 80.1 |
| EX 3 | 99.3 | 97.9 | 76.7 | 111.6 | 106.2 |
| CE A | 72.2 | 246.3 | 0.0 | 0.0 | 213.0 |
| CE B | 75.4 | 0.0 | 95.0 | 88.2 | 8.2 |
| CE C | 80.1 | 31.1 | 79.7 | 68.5 | 79.0 |
| CE D | 96.4 | 68.7 | 98.8 | 92.0 | 27.7 |
| CE E | 87.6 | 105.8 | 104.1 | 71.1 | 100.8 |
| CE F | 89.4 | 81.0 | 89.7 | 97.1 | 47.3 |

TABLE 7

| | Percent Recovery of E. coli (equation 1) | | | | |
|---|---|---|---|---|---|
| | Type of Antimicrobial Solution Used | | | | |
| | | Hydrogen | | | |
| Aqueous | Quat | Peroxide | Acid | Bleach | Peracetic |
| Composition of | Based | Based | Based | Based | Acid Based |
| EX 1 | 103.3 | 107.5 | 97.5 | 119.2 | 117.2 |
| EX 2 | 94.6 | 97.8 | 96.8 | 97.5 | 100.0 |
| EX 3 | 102.9 | 106.8 | 112.1 | 103.9 | 85.3 |
| CE A | 130.8 | 21.7 | 0.0 | 0.2 | 32.7 |
| CE B | 87.1 | 71.6 | 91.7 | 87.1 | 28.0 |
| CE C | 102.7 | 90.8 | 75.6 | 75.6 | 94.9 |
| CE D | 90.6 | 79.5 | 86.9 | 73.8 | 32.9 |
| CE E | 91.6 | 84.3 | 80.3 | 69.9 | 77.6 |
| CE F | 88.7 | 82.3 | 81.3 | 75.3 | 77.0 |

### Examples 9-12 (EX 9-EX 12)

Compositions of the disclosure (Examples 19-12) were individually prepared by mixing in sterilizable glass containers the ingredients shown in Table 8. The compositions were sterilized by autoclaving at 121° C. for 15 minutes.

TABLE 8
Aqueous Compositions (Examples 9-12)

| | Amounts of Ingredients in the Compositions | | | | | | | |
|---|---|---|---|---|---|---|---|---|
| | Example 9 | | Example 10 | | Example 11 | | Example 12 | |
| Ingredients | (EX 9) | | (EX 10) | | (EX 11) | | (EX 12) | |
| Soy Lecithin | 7 | g | 7 | g | 7 | g | 7 | g |
| Sodium | 15 | g | 15 | g | 15 | g | 15 | g |
| phosphate | | | | | | | | |
| dibasic | | | | | | | | |
| Potassium | 3 | g | 3 | g | 3 | g | 3 | g |
| phosphate | | | | | | | | |
| monobasic | | | | | | | | |
| Sodium pyruvate | 7.5 | g | 7.5 | g | 7.5 | g | 7.5 | g |
| Porcine peptone | 0.8 | g | 0.8 | g | 0.8 | g | 0.8 | g |
| Soytone | 0.8 | g | 0.8 | g | 0.8 | g | 0.8 | g |
| Tryptone | 0.8 | g | 0.8 | g | 0.8 | g | 0.8 | g |
| Ascorbic acid | 0.5 | g | 1 | g | 5 | g | 7.5 | g |
| Glycerol | 7.5 | mL | 7.5 | mL | 7.5 | mL | 7.5 | mL |
| Distilled Water | 992.5 | mL | 992.5 | mL | 992.5 | mL | 992.5 | mL |

### Example 13. Stability Determination of Aqueous Compositions Stored at 2-8° C.

A titration solution was freshly prepared at each test point by dissolving 33 mg of 2,6-dichlorophenolindophenol sodium salt dihydrate (DCPIP) in 100 mL of sterile, distilled water. The aqueous compositions of Examples 9-12 were freshly prepared and each composition was stored in a 10 mL sterile glass tube at 2-8° C. The compositions were measured for ascorbic acid content at the following timepoints: immediately after preparation (Day 0), 5 days after preparation (Day 5), and 11 days after preparation (Day 11). At each time point, a 0.5 mL aliquot was removed from each of the aqueous compositions and diluted in 15 mL of sterile water. Each diluted sample was titrated with the DCPIP titration solution with the titration end point being the detection of a faint blue color in the diluted sample. The appearance of the faint blue color indicated that all of the ascorbic acid in the sample had reacted with the DCPIP reagent. The amount of DCPIP titrated for each sample was recorded. The percent ascorbic acid remaining in a sample at each timepoint was calculated by comparing the amount of DCPIP titrated at a given time point versus the amount titrated on Day 0 (titrating immediately after preparation). The results are reported in Table 9 with the ascorbic acid measurement on Day 0 defined as 100% for each sample.

TABLE 9

| | |
|---|---|
| | Percent Ascorbic Acid Content in Aqueous |
| Aqueous | Compositions on Storage at 2-8° C. |

| Composition of | Day 0 | Day 5 | Day 11 |
|---|---|---|---|
| EX 9 | 100% | 91.7% | 83.3% |
| EX 10 | 100% | 115.4% | 96.2% |
| EX 11 | 100% | 104.7% | 87.8% |
| EX 12 | 100% | 101.9% | 94.8% |

### Example 14. Stability Determination of Aqueous Compositions Stored at Room Temperature

The same procedure as described in Example 13 was followed with the exception that the freshly prepared aqueous compositions of Examples 9-12 were stored at room temperature (23-25° C.), instead of 2-8° C. In addition, a solution of ascorbic acid in sterile, distilled water (0.5 g/L) was prepared as Comparative Example G (CE G). Comparative Example G was stored and analyzed using the same procedures as for Examples 9-12. The results are reported in Table 10.

TABLE 10

| | Percent Ascorbic Acid Content in Aqueous | | |
|---|---|---|---|
| Aqueous | Compositions on Storage at Room Temperature | | |
| Composition of | Day 0 | Day 5 | Day 11 |
| EX 9 | 100% | 75.0% | 41.7% |
| EX 10 | 100% | 96.2% | 76.9% |
| EX 11 | 100% | 94.6% | 77.0% |
| EX 12 | 100% | 99.5% | 90.0% |
| CE G | 100% | 32.5% | 17.4% |

1. A composition comprising
an ascorbic acid compound;
at least one compound capable of deactivating an antimicrobial agent;
a compound selected from the group consisting of pyruvate, at least one salt form thereof, and at least one hydrate of any of the foregoing;
sufficient water to form a solution or dispersion of all of the components of the composition.

2. The composition of claim 1 wherein the ascorbic acid compound is selected from the group consisting of ascorbic acid, 2-phospho ascorbic acid, 6-O-alkyl ascorbic acid, 2,6-di-O-alkenyl ascorbic acid, 3-O alkyl ascorbic acid, 2-O-α-glycopyranosyl ascorbic acid, glyceryl ascorbate, 5,5-di-O-alkyl ascorbic acid, at least one salt form of any of the foregoing, and at least one hydrate of any of the foregoing.

3. The composition of claim 1 wherein the ascorbic acid compound is selected from the group consisting of ascorbic acid, 2-phospho ascorbic acid, at least one salt form of any of the foregoing, and at least one hydrate of any of the foregoing.

4. The composition of claim 1 wherein the ascorbic acid compound is 2-phospho ascorbic acid, at least one salt form of thereof, or at least one hydrate of any of the foregoing.

5. The composition of claim 1 wherein the at least one compound capable of deactivating an antimicrobial agent comprises one or more peptones, the one or more peptones optionally comprising one or more of soytone, tryptone, and porcine peptone, and further optionally being a combination of soytone, tryptone, and porcine peptone.

6. The composition of claim 1 further comprising a polyol surfactant, wherein the polyol surfactant is optionally glycerol.

7. The composition of claim 1 further comprising an emulsifier.

8. The composition of claim 1 wherein after storage at 2-8 degrees C. for 1 week under ambient atmospheric conditions, the amount of ascorbic acid compound in the composition decreases by no more than 60% when compared to the amount of ascorbic acid compound present in the composition prior to storage at 2-8 degrees C. for 1 week.

9. The composition of claim 1 wherein the composition has a pH of about 6-8.

10. The composition of claim 1 further comprising a buffer.

11. The composition of claim 1 further comprising a buffer wherein the buffer has a concentration of about 10 mM to about 100 mM.

12. The composition of claim 1 comprising about 1-20 g/L of lecithin.

13. The composition of claim 1 comprising about 1-20 g/L of pyruvate based on the weight of sodium pyruvate.

14-15. (canceled)

16. A method of culturing for one or more microorganisms comprising
sampling a surface to remove a sample of the one or more microorganisms from the surface;
contacting the sample with a composition of claim 1;
contacting the sample with a growth media; and
allowing at least some of the one or more microorganisms to multiply within the growth media.

17. The method of claim 16 wherein the step of contacting the sample with a composition of any of the preceding claims occurs before the step of contacting the sample with a growth media.

18. The method of claim 16 wherein the step of contacting the sample with a composition of any of the preceding claims occurs after the step of contacting the sample with a growth media.

19. The method of claim 16 wherein the surface has at least one antimicrobial agent disposed thereon.

20. The method of claim 16 wherein the growth medium comprises agar.

21. The method of claim 16 wherein the growth medium is a thin film growth medium.

22. The method of claim 16 wherein the microorganism is selected from the group consisting of E. coli, S. typhi, L. monocytogenes, and a Salmonella microorganism, and mixtures thereof.
